(19) United States
(12) Patent Application Publication
(10) Pub. No.: US 2020/0005936 A1
(43) Pub. Date: Jan. 2, 2020

(54) SURGICAL INSTRUMENT SET AND INSTRUMENT MANAGEMENT SYSTEM

(71) Applicant: DGSHAPE Corporation, Hamamatsu-shi (JP)

(72) Inventor: Masaki HANAJIMA, Hamamatsu-shi (JP)

(21) Appl. No.: 16/452,619

(22) Filed: Jun. 26, 2019

(30) Foreign Application Priority Data
Jun. 27, 2018 (JP) 2018-121720

(51) Int. Cl. G16H 40/40

(52) U.S. Cl. CPC G16H 40/40; A61B 90/92

(57) ABSTRACT

A system includes a first memory storage that stores instrument IDs associated in a one-to-one correspondence with instruments or with instruments in a surgical instrument set. A second memory storage stores set IDs associated in a one-to-one correspondence with surgical instrument sets. A third memory storage stores work histories of the instruments identified by the instrument IDs, the work histories being stored in association with the instrument IDs. A fourth memory storage stores work histories of the surgical instrument sets identified by the set IDs, the work histories being stored in association with the set IDs. A first processor obtains a utilization rate of an instrument. A second processor obtains a utilization rate of a surgical instrument set.

### CROSS REFERENCE TO RELATED APPLICATIONS

This application claims the benefit of priority to Japanese Patent Application No. 2018-121720 filed on Jun. 27, 2018. The entire contents of this application are hereby incorporated herein by reference.

### BACKGROUND OF THE INVENTION

### 1. Field of the Invention

The present invention relates to a management system for surgical instrument sets and instruments.

### 2. Description of the Related Art

JP 2003-016198 A discloses a device that provides identification codes individually for small instruments used in medical consultation, surgical operation, and medical examination, and that reads the identification codes. Data about patients, data about doctors attending to the patients, inventory data of small steel instruments, and the like are stored as medical data in a database of the managing device. After a medical practice such as a surgical operation and a treatment is carried out for a patient, read data are compared with the medical data stored in the database of the managing device.

JP 2009-72338 A discloses a management system for a medical device that is cleaned after every use for future repeated use. JP 2009-72338 A discloses that a display part displays different colors according to the state of an endoscope.

JP 2012-215990 A discloses a device for supporting a picking work of medical instruments and materials. Specifically, the device includes a storage part that stores identifiers of medical instruments and materials and images of the medical instruments and materials, and another storage part that associates identifiers of mutually similar medical instruments and materials with each other and stores them. The device disclosed in JP 2012-215990 A is configured to be able to display an image of a medical instrument or material specified by the user and an image of medical instrument or material that is similar the specified medical instrument or material side by side.

JP 2013-116234 A discloses an apparatus that manages the locations of medical devices. The apparatus disclosed in the publication has a device information acquisition part that acquires device information including read information of identification tags attached to the medical devices from tag readers installed at a plurality of locations in a medical facility. A status holding part holds the statuses of the medical devices, which includes the locations of the medical devices that are identified by the acquired device information. A display control part causes a display to display the statuses of the medical devices held by the status holding part. The display control part also causes the display to hide the medical device in use for a medical practice among the medical devices.

JP 2015-197735 A discloses a device capable of distributing guidance information that helps general medical staff. The device disclosed therein uses medical work information that represents details of medical practices carried out by experienced medical staff as the guidance information. Then, the device distributes the guidance information in response to a request from general medical staff.

Japanese Registered Utility Model No. 3199614 discloses a surgical instrument management system. The system disclosed therein records photographs of external appearance related to surgical instruments. The system uses bar code information to create an asset number list of the surgical instruments prepared before a surgical operation. Next, the system reads identification patterns of the surgical instruments that were collected after the surgical operation. The system displays the external appearance photographs of the surgical instruments, and also determines whether or not the asset numbers of the surgical instruments collected after the surgical operation match the asset numbers of the surgical instruments prepared before the surgical operation.

### SUMMARY OF THE INVENTION

Generally, surgical instruments are managed after a surgical operation in the following sequence: collection, cleaning, assembling, sealing, sterilization, and storage. The instruments are sterilized and thereafter stored, and used again in another surgical operation. Medical facilities, such as hospitals, possess many types of instruments and surgical instrument sets, and they have a plurality of instruments and surgical instrument sets for each type. When in use, the stored instruments or surgical instrument sets are picked out each time. When there are many instruments of the same type or many surgical instrument sets of the same type, the management process is complicated and troublesome. For example, the frequency of use tends to vary among the instruments of the same type and among the surgical instrument sets of the same type.

A surgical instrument set and instrument management system in this disclosure includes, for example, a first memory storage, a memory second storage, a third memory storage, a fourth memory storage, a first processor, and a second processor.

The first memory storage stores instrument IDs associated in a one-to-one correspondence with instruments. The second memory storage stores set IDs associated in a one-to-one correspondence with surgical instrument sets each including a plurality of instruments. The third memory storage storing work histories of the instruments identified by the instrument IDs, the work histories stored in association with the instrument IDs. The fourth memory storage stores work histories of the surgical instrument sets identified by the set IDs, the work histories stored in association with the set IDs. The first processor is configured or programmed to obtain a utilization rate of an instrument based on the work history of the instrument stored in the third memory storage and a predetermined first calculation formula. The second processor is configured or programmed to obtain a utilization rate of a surgical instrument set based on the work history of the surgical instrument set stored in the fourth memory storage and a predetermined second calculation formula.

The preferred embodiments of the surgical instrument sets and instrument management systems herein are capable of obtaining the utilization rate of an instrument and the utilization rate of a surgical instrument set. This results in easy management of the instruments and the surgical instrument sets even when there are a large number of instruments of the same type and a large number of surgical instrument sets of the same type.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic view of a system 10.

FIG. 2 is a schematic view illustrating an example of an application of the system 10.

FIG. 3 illustrates an example of the configuration of a database 400 in which a first memory storage 101 and a second memory storage 102 are integrated into a single unit.

FIG. 4 illustrates an example of a table 420 that records work information obtained at a collection area A2.

FIG. 5 illustrates an example of a table 440 that records work information obtained at a sterilization area A5.

FIG. 6 illustrates an example of data obtained by extracting the work history of a certain instrument.

FIG. 7 illustrates an example in which the work histories of a plurality of instruments of the same type are extracted based on the type of instrument.

### DETAILED DESCRIPTION OF THE PREFERRED EMBODIMENTS

Hereinbelow, surgical instrument sets and instrument management systems according to preferred embodiments of the present invention will be described with reference to the drawings. It should be noted, however, that the preferred embodiments described herein are, of course, not intended to limit the present invention.

Herein, a surgical instrument set refers to a set of a plurality of medical instruments that are used in a surgical operation or in one of the steps of the surgical operation. Herein, the medical instruments may be referred to simply as “instruments”, as appropriate.

Examples of the instruments of the surgical instrument set include trocars, forceps, cutting devices, cleaning and suction devices, scissors (Cooper scissors), scalpels, scalpel holders, cannulae, thumb forceps, retractors, scales, sounds, elevators, raspatories, suction tubes, rib spreaders, rib contractors, needle holders, injection syringes, metal basins, pus basins, glasses, pins, mirrors, files, mouth gags, wound clips, handpieces, chisels, sharp spoons, dissectors, surgical needles, intervertebral disc rongeurs, water receivers, needles, depressors, bougies, ventilation tubes, bone impactors, Luer bone rongeurs, needle-nose pliers, hammers, goniometers, pipettes, enemators, and syringes.

The medical instruments may include an instrument including a plurality of components. For example, the instruments used in a laparoscopic surgery, such as trocars, forceps, a cutting device, and a cleaning and suction device, may include a plurality of components, and they are collected after the surgical operation and dissembled into a plurality of components. Herein, an element, portion or component that defines or makes up a medical instrument is referred to as a “component”. Moreover, a component may further be composed of a plurality of components. When this is the case, each of the components is also referred to as a “component”.

For the medical instruments and the components of the medical instruments, different cleaning procedures are specified. For this reason, the medical instruments and the components of the instruments are collected after a surgical operation, then sorted by specified cleaning procedures, and then sent to a cleaning step. After the cleaning, the instruments each composed of a plurality of components are assembled. Also, the medical instruments are classified by the type of surgical instrument set or by the type of medical instrument, and they are enclosed and sealed in predetermined containers. Thereafter, the instruments are sterilized in a specified sterilization method, and then stored until they are used next time. Thus, when using a surgical instrument set, the following sequence of steps, surgical operation→collection (sorting)→cleaning→assembling (sealing)→sterilization→storage→surgical operation, is repeated. It is important that the instruments and components of the surgical instrument set be properly cleaned in an appropriate method and procedure, in order to prevent hospital infection, for example.

Generally, after a surgical operation, the surgical instrument sets and the surgical instruments undergo the sequence of steps, collection, cleaning, assembling, sealing, sterilization, and storage. The instruments that are stored after sterilization are reused in another surgical operation. In such cases, the management process is complicated and troublesome. For example, the frequency of use varies among the instruments of the same type or among the surgical instrument sets of the same type. Moreover, it is difficult to judge how efficiently a surgical instrument set or a surgical instrument is used. Furthermore, even when there is a surgical instrument set or a surgical instrument that has not been used at all for a certain period of time, nobody may notice such a case.

The surgical instrument sets and instrument management systems described herein may be used to manage, for example, surgical instrument sets and instruments in an appropriate manner.

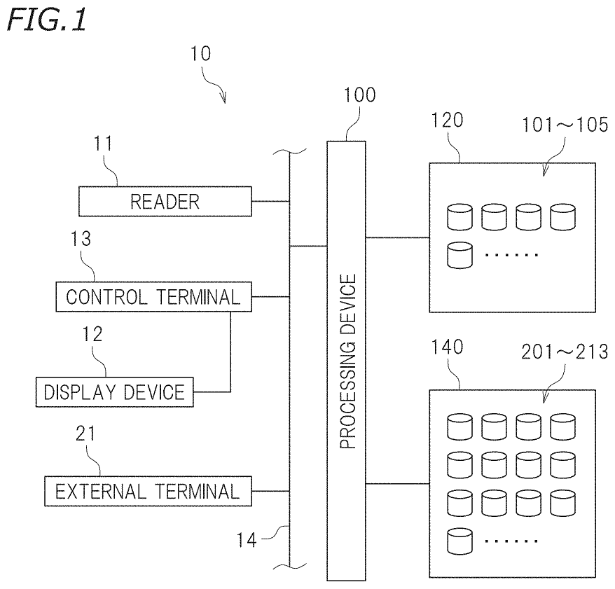

FIG. 1 is a schematic view of a surgical instrument set and instrument management system 10 (which may be simply referred to as a “system 10” when appropriate in this description). In the preferred embodiment shown in FIG. 1, the system 10 includes a reader 11, a display 12, a control terminal 13, and a processor 100, as the hardware configuration. However, the hardware configuration of the system 10 may not necessarily be limited to the preferred embodiment shown in FIG. 1. Various processes of the system 10 may be implemented by cooperative combinations of software and hardware in the control terminal 13 and the processor 100, for example.

As illustrated in FIG. 1, the system 10 proposed herein includes a first memory storage 101 to a fifth memory storage 105 and a first processor 201 to a thirteenth processor 213. In addition to the first memory storage 101 to the fifth memory storage 105 and the first processor 201 to the thirteenth processor 213 as will be described herein, the system 10 may include any additional storage and/or any additional processor as required.

Of these, the first memory storage 101 stores instrument IDs associated in a one-to-one correspondence with instruments or with a plurality of instruments included in a surgical instrument set.

The second memory storage 102 stores set IDs associated in a one-to-one correspondence with surgical instrument sets.

The third memory storage 103 stores work histories of the instruments identified by the instrument IDs, which are stored in association with the instrument IDs.

The fourth memory storage 104 stores work histories of the surgical instrument sets identified by the set IDs, which are stored in association with the set IDs.

The first processor 201 is configured or programmed to obtain the utilization rate of an instrument based on the work history of the instrument stored in the third memory storage 103 and a predetermined first calculation formula.

The second processor 202 is configured or programmed to obtain the utilization rate of a surgical instrument set based on the work history of the surgical instrument set stored in the fourth memory storage 104 and a predetermined second calculation formula.

The system 10 as described above is capable of obtaining the utilization rate of an instrument and the utilization rate of a surgical instrument set. This results in easy management of the instruments and the surgical instrument sets even when there are many instruments of the same type and many surgical instrument sets of the same type. For example, the instruments and the surgical instrument sets that show a low utilization rate should be used preferentially so that variations in the frequency of use can be reduced among the instruments and the surgical instrument sets of the same type. Furthermore, because the system 10 obtains the utilization rates of the instruments and the utilization rates of the surgical instrument sets, it is easy to recognize the presence of a surgical instrument set or a surgical instrument that has not been used at all for a certain period.

The system 10 suitably records the work history of the instrument identified by an instrument ID and the work history of the surgical instrument set identified by a set ID in the following steps, surgical operation→collection (sorting)→cleaning→assembling (sealing)→sterilization→storage→surgical operation, for example. The system 10 is configured or programmed to be able to obtain the utilization rate of the instrument based on the work history of the instrument and obtain the utilization rate of the surgical instrument set based on the work history of the surgical instrument set. Hereinafter, an example of an application of the system 10 will be illustrated.

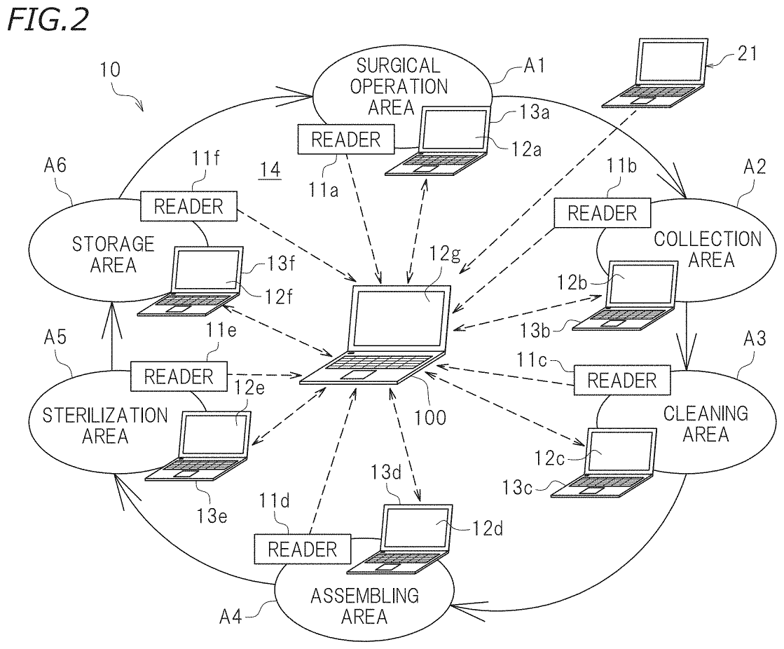

FIG. 2 is a schematic view illustrating an example of an application of the system 10. The present preferred embodiment shown in FIG. 2 illustrates an example in which control terminals 13a to 13f and readers 11a to 11f are arranged respectively in areas A1 to A6, which are a surgical operation area A1, a collection area A2, a cleaning area A3, an assembling area A4, a sterilization area A5, and a storage area A6.

The surgical operation area A1 is an area in which the instruments and the surgical instrument sets are used by doctors and nurses, and an area in which a surgical operation and a medical examination are carried out.

The collection area A2 is an area in which the surgical instrument sets and the surgical instruments are collected from the surgical operation area A1. Here, the surgical instruments may be collected separately, surgical instrument by surgical instrument.

The cleaning area A3 is an area in which the surgical instrument sets and the surgical instruments are cleaned. Here, the surgical instruments may be disassembled into components for cleaning.

The assembling area A4 is an area in which the cleaned surgical instrument sets and the cleaned surgical instruments are assembled and packaged into predetermined containers or bags.

The sterilization area A5 is an area in which the surgical instrument sets and the surgical instruments are sterilized. The surgical instrument sets and the surgical instruments are sterilized as they are kept packaged in predetermined containers or bags.

The storage area A6 is an area in which the sterilized surgical instrument sets and the sterilized surgical instruments are stored.

Note that this preferred embodiment includes the surgical operation area A1, the collection area A2, the cleaning area A3, the assembling area A4, the sterilization area A5, and the storage area A6, but this is merely illustrative. For example, in some cases, the surgical instrument sets and the surgical instruments may need repair or maintenance. In such cases, it is possible to provide the concept of a maintenance area so that the surgical instrument sets and the surgical instruments may be sorted into the maintenance area when they are sent to repair or maintenance. In addition, the processes such as cleaning, assembling, and sterilization may be carried out by a specialist contractor, unlike surgical operations. In such cases, the cleaning area A3, the assembling area A4, and the sterilization area A5 may be provided in a different facility from the facility that contains the surgical operation area A1, the collection area A2, and the storage area A6. It is also possible that the surgical operation area A1, the collection area A2, and the storage area A6 may be provided in different locations. In addition, the system 10 is able to collectively manage the surgical instrument sets and the instruments that are used in a plurality of hospitals. In such cases, the surgical operation area A1, the collection area A2, and the like may be provided in a plurality of locations.

Each of the above-described areas A1 to A6 may be defined as a location where a predetermined work is performed. However, in the system, it is desirable that respective identifiers (IDs) be assigned in association with the areas A1 to A6, and they may be stored in master data. In the system, the readers 11a to 11f and the control terminals 13a to 13f, which are disposed in the respective areas A1 to A6, may be associated respectively with the areas A1 to A6. For example, it is possible that the information that is input with the readers 11a to 11f or with the control terminals 13a to 13f may be handled by the system 10 as the information that is input at the respective areas A1 to A6.

It is also possible that the actual locations of the areas A1 to A6 may be overlapped with each other. For example, when collection is carried out at the same location as where a surgical operation or a medical practice is carried out, the location of the surgical operation area A1 and the location of the collection area A2 may be overlapped with each other. In addition, it is also possible that the reader 11, the display 12, and the control terminal 13 may be used in common between the surgical operation area A1 and the collection area A2. In this case, it is preferable that the functions and settings of the control terminal 13 be switched from one to another so as to be suitable for the areas A1 to A6 by, for example, a process on software. For example, the control terminal 13 may be configured to be able to switch between the functions and settings suitable for the surgical operation area A1 and the functions and settings suitable for the collection area A2, as appropriate, by a predetermined operation.

As illustrated in FIG. 2, the system 10 is implemented by the processor 100, the control terminals 13a to 13f, and the readers 11a to 11f. The control terminals 13a to 13f and the readers 11a to 11f are disposed respectively in the areas A1 to A6. The control terminals 13a to 13f and the processor 100 respectively include displays 12a to 12g. In the present description, the reader(s), the display(s), and the control terminal(s) may be simply and collectively referred to as the “reader 11”, the “display 12”, and the “control terminal 13” when it is unnecessary to make a distinction between the readers 11a to 11f, between the displays 12a to 12g, and between the control terminals 13a to 13f.

Referring to FIG. 2, one reader 11, one display 12, and one control terminal 13 are arranged in each of the areas A1 to A6. However, it is also possible that a plurality of readers 11, a plurality of displays 12, and a plurality of control terminals 13 may be provided in each of the areas A1 to A6. Also, a reader 11, a display 12, and a control terminal 13 may be used in common for different areas among the areas A1 to A6. It should be noted that FIGS. 1 and 2 are schematic views of the system 10. However, the system 10 is not limited to such a preferred embodiment. The system 10 may further include further larger numbers of readers 11, displays 12, and control terminals 13.

Each of the readers 11a to 11f disposed in the respective areas A1 to A6 is a device that reads item-identifying information of an instrument included in a surgical instrument set, item-identifying information incorporated into a component of the instrument, or item-identifying information attached to a bag or a container that encloses the instrument or the surgical instrument set.

Here, the item-identifying information may be a two-dimensional symbol in a predetermined form. Examples of the item-identifying information used for the instruments in a surgical instrument set may include two-dimensional bar codes, contactless tags such as RFIDs, and engraved marks formed in the instrument surfaces. The engraved marks may be formed by laser engraving or impact engraving, for example.

Formation of the two-dimensional symbol by impact engraving may be performed by using an impact printer (e.g., MPX-95 manufactured by Roland DG Corp.), for example. Such an impact printer is capable of forming a data matrix with a very small size of, for example, about 1 mm square to about 4 mm square. The impact-engraved two-dimensional symbol is formed by indenting the surface of the instrument. The impact-engraved two-dimensional symbol is unlikely to damage, for example, a plated coating film on the surface of the instrument, so the impact-engraved two-dimensional symbol does not cause the instrument to rust easily. Moreover, due to the development of impact printer or the like, impact engraving of two-dimensional symbols can be applied to the existing instruments on which no two-dimensional symbol has been formed, and two-dimensional symbols can be set by the user and formed on the existing instruments.

In the assembling area A4, the sterilization area A5, or the storage area A6, each of the instruments is individually packaged appropriately, and each of the surgical instrument sets is collectively enclosed in a bag or a container, for example. In this case, each of the bags or the containers for enclosing an instrument or a surgical instrument set may be provided with item-identifying information, such as a two-dimensional symbol.

Each of the readers 11a to 11f may include a reader, detector or sensor to detect the two-dimensional symbol. Examples of the reader, detector or sensor to detect the two-dimensional symbol include cameras and CCD image sensors. The item-identifying information that is assigned to the instrument or the component may be read by causing the item-identifying information (for example, an impact-engraved two-dimensional symbol) attached to the instrument or the component to be aimed to a desired one of the reading units of the readers 11a to 11f. When the instrument or the surgical instrument set is enclosed in a bag or a container, the item-identifying information may be read by aiming the item-identifying information attached on the bag or the container to a desired one of the readers 11a to 11f.

Although each of the control terminals 13a to 13f and the processor 100 is depicted as a laptop terminal in FIG. 2, the type of terminal is not limited thereto. Each of the terminals may be a tablet terminal or a desktop terminal, for example. The control terminals 13a to 13f may be configured or programmed to cooperate with the processor 100 to implement various processes of the system 10. In this preferred embodiment, the processor 100 may function as a host computer that serves as a backbone of the processes of the system 10. Each of the control terminals 13a to 13f may cooperate with the processor 100 and function as a client computer of the system 10. The processor 100 and the control terminals 13a to 13f include memory storage devices, and the memory storage devices of the processor 100 and the control terminals 13a to 13f may be connected to each other via a communication network 14. The memory storage devices of the processor 100 and the control terminals 13a to 13f may be configured so that information can be written into and retrieved from the memory storage devices.

Each of the control terminals 13a to 13f and the processor 100 includes an arithmetic device [also referred to as a processor, CPU (central processor), or MPU (micro-processor)] and a memory storage device (such as a memory and a hard disk). The functions of the control terminals 13a to 13f and the processor 100 are embodied by cooperative combinations with software stored in the memory storage device. For example, the configurations and the processes of the control terminals 13a to 13f and the processor 100 may be embodied as processing modules that perform predetermined computing tasks, data, or a portion thereof. Specifically, each of the processing modules may be a program or programs executable by a computer or processor.

Referring to FIG. 1, each of the processor 100 and the control terminals 13a to 13f includes a memory storage area 120 for storage of data and a memory storage area 140 for storage of processing modules.

The memory storage area 120 for storage of data stores various kinds of master data and various data acquired by the readers 11a to 11f and the control terminals 13a to 13f, in addition to the above-described memory storages 101 to 105.

The memory storage area 140 for storage of processing modules stores processing modules of the system 10 as appropriate. The processing modules stored in the memory storage area 140 are not limited to the foregoing processors 201 to 213. The processor 100 and the control terminals 13a to 13f may be configured to, for example, call and execute the processing modules stored in the memory storage area 140. Herein, the memory storage area 120 that stores data may be constructed by, for example, a separate data server that is independent of the processor 100 and the control terminals 13a to 13f.

Each of the control terminals 13a to 13f and the processor 100 may be embodied by a single device, or may exhibit the functions as a processor by cooperative combinations of a plurality of devices. That is, although FIG. 1 depicts that the processor 100 includes one single device, the processor 100 is not limited to being one single device.

The control terminals 13a to 13f and the processor 100 respectively include displays 12a to 12g, each of which functions as a display. The displays 12a to 12g are configured to display images according to programs incorporated in the control terminals 13a to 13f and the processor 100. Each of the displays 12a to 12g may include a touchscreen. When each of the displays 12a to 12g includes a touchscreen, the system 10 may be operated by the user's touching the screen of the display 12, for example.

The readers 11a to 11f, the control terminals 13a to 13f, and the processor 100 may be bidirectionally date-communicably connected to each other. In the preferred embodiment shown in FIG. 2, the readers 11a to 11f, the control terminals 13a to 13f, and the processor 100 cooperate with each other in such a state as to be capable of performing data communication with each other through the communication network 14. It is desirable that the control terminals 13a to 13f, the readers 11a to 11f, and the processor 100 be connected in such a state as to be able to make information communication with each other via, for example, a wireless communication network through a router (not shown) or a wired communication network through LAN cables.

Each of the displays 12a to 12f of the control terminals 13a to 13f shown in FIG. 2 functions as the display 12 shown in FIG. 1. In the preferred embodiment shown in FIG. 2, the control terminals 13a to 13f and the processor 100 are cooperated with each other to function as the processor 100 of the system 10 (see FIG. 1). In the preferred embodiments shown in FIGS. 1 and 2, the system 10 is embodied by a plurality of devices. In the system 10, a plurality of personal computers, such as the control terminals 13a to 13f and the processor 100, cooperate with each other. The system 10 may be used simultaneously in parallel at a plurality of remote locations by a plurality of different users. A large number of surgical instrument sets and a large number of instruments may be managed in parallel in a plurality of steps.

As illustrated in FIG. 1, the system 10 includes memory storages 101 to 105, namely, the first memory storage 101 to the fifth memory storage 105, and processors 201 to 213, namely, the first processor 201 to the thirteenth processor 213.

In the system 10, the processes performed by the processors 201 to 213 may also be processed by any of the processor 100 and the control terminals 13a to 13f. The information stored in the memory storages 101 to 105 may be managed by the processor 100 as a host computer so as to be shared by the control terminals 13a to 13f.

The first memory storage 101 stores instrument IDs associated in a one-to-one correspondence with instruments or with a plurality of instruments included in a surgical instrument set. Specifically, in the system 10, the instruments included in the surgical instrument set are provided with respective instrument IDs. Each of the instrument IDs corresponds in a one-to-one correspondence to a specific instrument. As mentioned above, each of the instruments is provided with item-identifying information that is readable by the readers 11a to 11f. In this case, each of the instrument IDs may be associated with the item-identifying information provided to the respective instruments. The system 10 may be configured to be able to identify the instrument ID associated in a one-to-one correspondence with the instrument when the item-identifying information is read by one of the readers 11a to 11f.

Each of the instruments may further include a plurality of components. In the system 10, the components of each of the instruments are also provided with component IDs. Each of the component IDs corresponds in a one-to-one correspondence to a specific instrument and a component of the instrument. In the system 10, the instruments of a surgical instrument set and the components of an instrument may be identified also with component IDs. In this case, it is desirable that each of the components should be also provided with item-identifying information. It is desirable that the system 10 be configured so that when the item-identifying information is read by one of the readers 11a to 11f, the system 10 can identify the component ID and the instrument ID that are associated in a one-to-one correspondence with each of the components.

The second memory storage 102 stores set IDs associated in a one-to-one correspondence with surgical instrument sets. In other words, in this system 10, each of the surgical instrument sets is provided with a set ID that is associated in a one-to-one correspondence therewith, and a surgical instrument set is identified by a set ID.

In this preferred embodiment, the set ID associated in a one-to-one correspondence with a surgical instrument set, the instrument IDs of the instruments that are included in the surgical instrument set, and the component ID of the components that are included in the surgical instrument set are associated with each other when they are stored. In other words, when a set ID is identified, the instrument IDs of the instruments and the component IDs of the components that are included in the surgical instrument set identified by that set ID are identified accordingly. Also, when an instrument ID is identified, the set ID of the surgical instrument set to which the instrument belongs is identified accordingly. In cases where the instrument includes components, the component IDs of the components included in the instrument are identified from the instrument ID. Also, when a component ID of a component is identified, the instrument ID of the instrument that includes the component is identified accordingly. Moreover, when the instrument is included in a surgical instrument set, the set ID of the surgical instrument set that includes the instrument is identified accordingly.

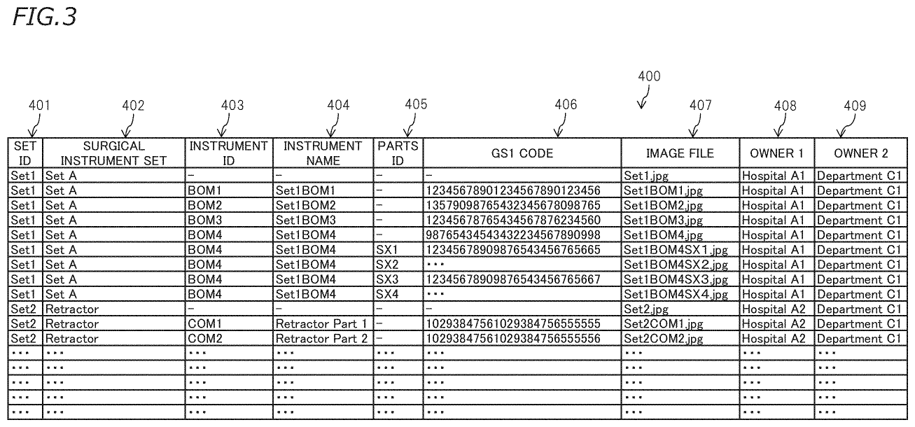

The first memory storage 101 and the second memory storage 102 described above may be include or be defined by respective independent databases. Alternatively, the first memory storage 101 and the second memory storage 102 may be a single database. FIG. 3 illustrates an example of the configuration of a database 400 in which the first memory storage 101 and the second memory storage 102 are integrated into a single database. FIG. 3 shows that there are provided a column 401 that stores set IDs, a column 402 that stores surgical instrument set names; a column 403 that stores instrument IDs; a column 404 that stores instrument names; a column 405 that stores component IDs; a column 406 that stores GS1 codes as item-identifying information; a column 407 that stores image files of surgical instrument sets, instruments, and components; and columns 408 and 409 that store information related to owners of instruments and surgical instrument sets. These IDs and pieces of information are stored in association with each other. The image files contain images of surgical instrument sets, instruments, and components. The image files may be configured so that the images are displayed appropriately on each of the displays 12a to 12g.

As in the preferred embodiment shown in FIG. 3, for the instruments included in the surgical instrument sets, the first memory storage 101 may additionally store set IDs of the surgical instrument sets respectively associated with the instrument IDs. As a result, when the instrument ID of an instrument included in a surgical instrument set is identified, the set ID of the surgical instrument set that includes the instrument is accordingly identified.

In addition, information related to owners of the instruments or the surgical instrument sets is stored in association with the instrument IDs and the set IDs. Herein, each of the owners may be specified by a department, such as surgery and internal medicine in cases of hospitals, for example. Moreover, when the instruments and the surgical instrument sets of a plurality of hospitals need to be managed collectively, the owner information may include information indicating a hospital, for example. In this way, the owner information may include a plurality of types of information at some hierarchical levels. For example, in this preferred embodiment, owner 1 includes information indicating a hospital, and owner 2 includes information indicating a medical department.

As illustrated in FIG. 3, it is desirable that the system 10 include pre-stored master data, including set IDs, instrument IDs, and component IDs, as well as associated information therewith, such as item-identifying information, image data, and information related to owners. The information that is stored in the master data is not limited to the items listed in FIG. 3. In addition, the master data in the system 10 may also include worker IDs, which may be assigned to name tags or the like distributed to workers. The worker IDs assigned to the name tags may be readable with the reader 11b.

Each of the readers 11a to 11f, the control terminals 13a to 13f, and the processor 100 may also be provided with item-identifying information assigned thereto, which may also be stored in advance in the master data of the system 10.

Moreover, information indicating cleaning method, packaging material, sterilization method, and the like may also be stored in advance appropriately in the master data of the system 10.

The third processor 203 is configured or programmed to extract an instrument ID of an instrument or a set ID of a surgical instrument set, for each type of instrument, each type of surgical instrument set, or each owner. This makes it possible to obtain the amount of stock and the utilization rate of an instrument or a surgical instrument set, for each instrument, each surgical instrument set, or each owner.

In this preferred embodiment, the readers 11a to 11f are arranged respectively at the areas A1 to A6, each being a predetermined work space. Each of the instruments includes item-identifying information. The first memory storage 101 stores the item-identifying information, the instrument ID, and the set ID of the instrument in association with each other.

In this preferred embodiment, the thirteenth processor 213 identifies the instrument ID and the set ID from item-identifying information when the item-identifying information is detected by the readers 11a to 11f. The thirteenth processor 213 is configured or programmed to add the information on the work that was performed for the instrument to the work history of the instrument in association with the identified instrument ID. The thirteenth processor 213 is also configured or programmed to add the information on the work that was performed for the surgical instrument set to the work history of the surgical instrument set in association with the identified set ID. Because the instrument ID and the set ID are identified by item-identifying information when the item-identifying information is detected by the readers 11a to 11f, it is unlikely that the work information is incorrectly input for a wrong instrument or a wrong surgical instrument set.

When item-identifying information is detected by the readers 11a to 11f disposed respectively in the areas A1 to A6, the instrument ID is identified by the item-identifying information, and the information on the work performed for the instrument in each of the areas A1 to A6 is added to the work history of the instrument. Also, when the set ID is identified by the item-identifying information, the information on the work performed for the surgical instrument set in each of the areas A1 to A6 is added to the work history of the surgical instrument set. Because the instrument or the surgical instrument set is identified through the readers 11a to 11f in this way, the input of the work history of the instrument or the surgical instrument set is carried out more reliably. Moreover, the work history of the instrument or the surgical instrument set records the time at which item-identifying information is detected by the readers 11a to 11f, in addition to the information on the work. Furthermore, it is also possible to add the information on the worker, the instruments and the materials used for the work, and the like, to the work history.

Herein, an example is shown that when item-identifying information is detected, the instrument ID is identified by the item-identifying information, and the information on the work performed for the instrument in the areas A1 to A6 is added to the work history of the instrument. However, the method and device to store the work history of the instrument identified by an instrument ID in association with the instrument ID and the specific the method and device to store the work history of the surgical instrument set identified by a set ID in association with the set ID are not limited to the above-described preferred embodiment, unless specifically stated otherwise. For example, it is also possible that the worker may manually input the information on the work at each of the areas A1 to A6.

In the surgical operation area A1, for example, when a stored instrument or a stored surgical instrument set is delivered into the surgical operation area A1, item-identifying information is detected by the reader 11a and the instrument ID or the set ID is identified. Then, the information indicating the date on which the instrument or the surgical instrument set is delivered and the information during use are recorded in association with the instrument ID or the set ID. The work information stored in the surgical operation area A1 may include, for example, the users (workers) such as doctors, patients, medical record numbers of the patients, surgical operations, and consultation numbers. This enables the system 10 to store the work history indicating what kind of surgical operation the instrument or the surgical instrument set was used for. As a result, the amount of information that can be tracked by the work history of the instrument or the surgical instrument set increases. It is also possible that the system 10 may import information contained in an existing electronic patient record into the work information obtained at the surgical operation area A1. It is also possible that the instrument ID or the set ID may be written into the existing electronic patient record. In this way, the systems may be mutually constructed so that the existing electronic patient record and the system 10 cooperate with each other.

The instruments and the surgical instrument sets used that were used in the surgical operation area A1 are brought into the collection area A2. In the collection area A2, the item-identifying information is detected by, for example, the reader lib, and the instrument IDs and the set IDs are identified. The work information stored in the collection area A2 may include information such as the date of collection and the worker ID, for example.

In the collection area A2, it is desirable that the display 12b display a list of the instruments included in a surgical instrument set, for example. It is desirable that the instruments be collected while the reader lib is detecting the item-identifying information of the instruments included in the surgical instrument set. When the item-identifying information of the instruments included in the surgical instrument set is detected, the instruments can be identified on the display 12b. It is also possible that, when the worker specifies an instrument displayed on the display 12b in the collection work, the display 12b may dim the indication of the instrument for which the item-identifying information was detected from the list of the instruments included in the surgical instrument set. In this case, the display 12b preferably includes a touchscreen. The display 12b may be configured so that, when the worker touches an indication representing the instrument to be collected (in other words, the instrument identified by the item-identifying information) from the list of the instruments displayed on the display 12b, the indication of the instrument is changed. It is desirable that, by such a process, the worker should be allowed to easily recognize visually that the subject instrument has been collected properly. Then, when all the instruments included in the surgical instrument set used in the surgical operation area A1 have been collected in the collection area A2, the collection of the surgical instrument set is completed. At that time, it is desirable that the worker should be allowed to easily recognize visually that the subject instruments have been collected properly by, for example, dimming the indication of the subject surgical instrument set or displaying an icon indicating the completion of collection on the display 12b.

In the cleaning area A3, the instrument ID is identified when the item-identifying information of an instrument included in a surgical instrument set is detected by, for example, the reader 11c. It is desirable that the work information obtained at the cleaning area A3 be stored in association with the identified instrument ID. The work information obtained at the cleaning area A3 may include, for example, a worker ID and information on the cleaning method such as the cleaning machine and the detergent that are used for the cleaning.

It is also desirable that the instrument ID and a cleaning method be associated with each other and stored in advance in the system 10. In this case, it is possible that when the reader 11c detects the item-identifying information of an instrument included in a surgical instrument set, the display 12c in the cleaning area A3 may display a cleaning process required for the instrument on the screen based on the identified instrument ID. Because the instrument ID is identified by the reader 11c, it is unlikely that the cleaning process is performed for a wrong instrument or that the work information is input for a wrong instrument. The reader 11c may be provided for, for example, a cleaning machine. In that case, it is also possible that, while the reader 11c is reading the item-identifying information of the instrument, the instrument may be put into the cleaning machine. It is desirable that the information on the cleaning conditions and the like that are set for the cleaning machine be recorded in the work information. Herein, examples of the cleaning method include manual cleaning, automated single-bath cleaning, automated multi-bath cleaning, ultrasonic cleaning, and chemical cleaning. Each of the cleaning methods may use a predetermined cleaning machine and a predetermined detergent in a predetermined way.

In addition, information contained in an existing electronic patient record may be imported into the work information obtained at the surgical operation area A1. In that case, the cleaning method and the sterilization method for the instrument are preferably recorded in the master data in advance, in association with information about patients and surgical operations. Accordingly, the system 10 may be constructed to appropriately identify a cleaning method required for the instrument based on the master data and the information about patients and surgical operations that is contained in the work information obtained at the surgical operation area A1. In this way, the system 10 may be constructed so as to identify a special cleaning method when such a special cleaning method is required by the instrument or the surgical instrument set based on the information provided by the electronic patient record. Likewise, in the sterilization area A5, the system 10 may be constructed so as to identify a special sterilization method when such a special sterilization method is required by the instrument or the surgical instrument set based on the information provided by the electronic patient record.

In the assembling area A4, various activities are performed, such as assembling of an instrument from cleaned components, and collecting of instruments to be included in a surgical instrument set to form the surgical instrument set. In assembling the instrument, required lubricating oil may be applied to the instrument.

In the assembling area A4, when the item-identifying information of an instrument included in a surgical instrument set is detected by the reader 11d, for example, the instrument ID of the instrument and the set ID of the surgical instrument set are identified. Then, the list of the instruments included in the surgical instrument set is displayed. At this time, it is desirable that the necessary assembling work that needs to be performed in the assembling area A4 be displayed on the screen. For example, it is possible to display the lubricating oil to be applied at the time of assembling of the instrument when such is specified in advance.

Each of the instruments and the surgical instrument sets is enclosed in a predetermined bag or container. At that time, the packaging material and the packaging method may be recorded additionally. Examples of the packaging material include nonwoven fabric, sterilization bags, containers, and trays. Examples of the packaging method include wrapping the instrument or the surgical instrument set with nonwoven fabric, enclosing the instrument or the surgical instrument set in a sterilization bag, and enclosing the instrument or the surgical instrument set that is wrapped with nonwoven fabric in a sterilization bag. The surgical instrument set should be packaged while checking that all the instruments to be included in the surgical instrument set are present. In this case, it is desirable that the instruments be packaged while displaying the list of instruments to be included in the surgical instrument set on the display 12d displays and detecting the item-identifying information of the instruments with the reader 11d when packaging the instruments. Also, it is desirable to change the indication of the instrument that has already been packaged in the list of the instruments. When the indication of the packaged instrument changes on the display 12d, the worker is able to easily confirm the instruments that have not yet been packaged. This prevents an instrument from being omitted from the surgical instrument set, and prevents a wrong instrument from being included in the surgical instrument set.

The instruments and the surgical instrument sets are packaged, for example, in a bag or a container and hermetically sealed therein. The instruments that are not included in surgical instrument sets are packaged piece by piece independently. The surgical instrument set is packaged, for example, in a single collection of instruments. Each of the packaged bags or the packaged containers may be provided with item-identifying information that identifies the bag or the container. Then, the item-identifying information attached to the bag or the container may be stored in association with the instrument ID for identifying the instrument enclosed therein and the set ID for identifying the surgical instrument set enclosed therein. For example, in the packaging work, an instrument or a surgical instrument set is placed into a bag or a container while reading the item-identifying information attached to the bag or the container and the item-identifying information of the instrument or the surgical instrument set to be enclosed in the bag or the container with the reader 11d. At this time, it is desirable that the system 10 store the item-identifying information of the bag or the container in association with the item-identifying information of the instrument or the surgical instrument set that is enclosed therein. This makes it possible to identify the set ID of the surgical instrument set or the instrument ID of the instrument that is enclosed in the bag or the container in a post-process, based on the item-identifying information attached on the bag or the container.

Thus, in the assembling area A4, the work information obtained at the assembling area A4 is stored in association with the item-identifying information of the bag or the container, for example, which is read by the reader 11c. The work information obtained at the assembling area A4 may include the instrument ID of the instrument enclosed in a bag or a container, the set ID of the surgical instrument sets enclosed in a bag or a container, the date and time at which item-identifying information is read by the reader 11c in assembling work, and the worker ID for identifying the worker. Moreover, it is desirable that the item-identifying information of the bag or the container and the item-identifying information of the instrument or the surgical instrument set enclosed therein be stored in association with each other.

In the sterilization area A5, the instruments and the surgical instrument sets are kept enclosed in a packaging material, such as a bag or a container, while they are handled. In the sterilization area A5, the item-identifying information attached to the bag or the container is read by, for example, the reader 11e. Then, the instrument or the surgical instrument set is identified based on the item-identifying information. Then, a predetermined sterilization treatment is performed for the instrument or the surgical instrument.

In this preferred embodiment, a database is prepared in advance in which a sterilization treatment is pre-stored in association with the instrument ID or the set ID. In the sterilization area A5, the item-identifying information attached to the bag or the container is read by the reader 11e, and the instrument ID or the set ID of the instrument or the surgical instrument set that is enclosed in the bag or the container is identified. The system 10 may be configured or programmed so that, based on the instrument ID or the set ID, the sterilization treatment to be performed at the sterilization area A5 is specified and displayed on the display 12e.

This preferably serves to allow the worker to easily understand the sterilization treatment that is to be performed for the instrument or the surgical instrument set enclosed in a bag or a container. When this is the case, for example, in the sterilization area A5, the reader 11e may be installed additionally on a sterilizer, such as an autoclave. In that case, the instrument or the surgical instrument set may be placed into the sterilizer after the item-identifying information attached to the bag or the container is read by the reader 11e. This preferably serves to allow the sterilization treatment to be performed under predetermined conditions (such as temperature and treatment time). In addition, the system 10 may be configured or programmed to prevent the worker from making errors by, for example, sounding an alarm in cases where the sterilizer in which the instrument or the surgical instrument set is to be placed is inappropriate or the conditions of the sterilization treatment that are set in the sterilizer are inappropriate when the item-identifying information attached to the bag or the container is read by the reader 11e.

In the sterilization area A5, the item-identifying information attached to the bag or the container is read by, for example, the reader 11e. Then, the instrument ID or the set ID is identified in association with the item-identifying information attached to the bag or the container. Then, the work information obtained at the sterilization area A5 is stored in association with the instrument ID, the set ID, and the item-identifying information attached to the bag or the container. The work information obtained at the sterilization area A5 may include, for example, the sterilization method and conditions of the sterilization treatment. Specifically, the work information obtained at the sterilization area A5 may further include, for example, information indicating the sterilizer used for the sterilization treatment, conditions of the sterilization such as temperature and treatment time, the end date and time of the sterilization treatment, the worker, and the like.

The storage area A6 is an area in which the sterilized instruments and the sterilized surgical instrument sets are stored. In the storage area A6, the item-identifying information attached to each of the bags and the containers is read by, for example, the reader 11f. Then, the instrument ID or the set ID is identified in association with the item-identifying information attached to each of the bags and the containers. After the instrument IDs of the enclosed instruments and the set IDs of the enclosed surgical instrument sets are identified, the bags and the containers are sorted and stored in an appropriate location. The work information recorded at the collection area A6 may include information indicating the storage location of each of the instruments and the surgical instrument sets, for example. Because the storage location of the instrument or the surgical instrument set are recorded, it is easy to pick out the instrument or the surgical instrument set.

As described above, the items of the work information to be recorded are different among the areas A1 to A6. The memory storage area 140 of the system 10 may include, for example, tables each in a predetermined format for storing the items of work information obtained at each of the areas A1 to A6. The items of the work information obtained at the areas A1 to A6 may be stored in the fields of the tables respectively prepared for the areas A1 to A6.

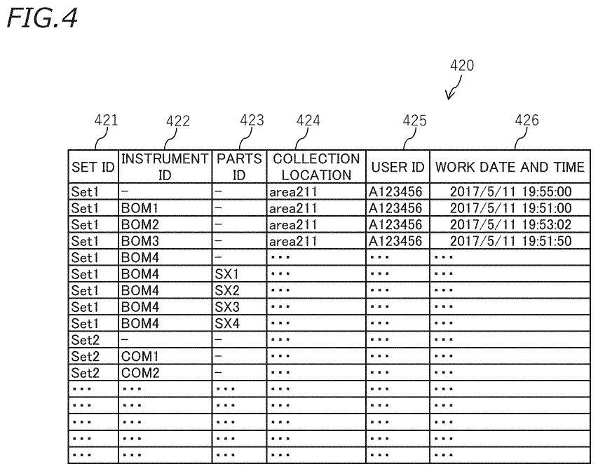

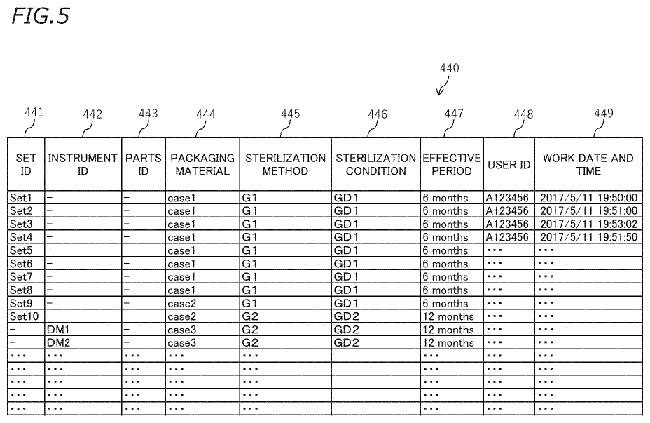

FIG. 4 illustrates an example of a table 420 that records work information obtained at the collection area A2. FIG. 5 illustrates an example of a table 440 that records work information obtained at the sterilization area A5.

In the collection area A2, instruments included in surgical instrument sets are collected from the surgical operation area A1 separately, for example.

In the collection area A2, each of the instruments may be collected while identifying the location of collection and the worker and reading the item-identifying information attached to the instrument with the reader 11b. Thus, as illustrated by the table 420 shown in FIG. 4, information is input into columns 421 to 426, which store set ID, instrument ID, component ID, collection location, worker, and work date and time, respectively. Note that the records of the work information to be recorded at the collection area A2 are not limited to the example shown in FIG. 4. The records of the work information to be recorded at the collection area A2 may include further detailed information.

In the sterilization area A5, the item-identifying information attached to a bag or a container is read by the reader 11e, for example, to thereby identify the instrument ID of the instrument or the set ID of the surgical instrument set that is enclosed in the bag or the container. Then, in the sterilization area A5, the sterilization method and the sterilization conditions required for the surgical instrument set or the instrument identified by the set ID or the instrument ID are set for the sterilizer. Then, the surgical instrument set or the instrument should be placed into the sterilizer while causing the reader 11e to read the item-identifying information attached to the bag or the container. For example, in the sterilization treatment, it is often the case that an indicator showing whether appropriate sterilization was performed may be fitted onto the packaging material that encloses the instrument or the surgical instrument set. In that case, the indicator may change its color, for example, under a predetermined condition. In the sterilization area A5, the indicator may be fitted onto the instrument or the surgical instrument set. The fitting of the indicator may also be carried out in the assembling area A4. The item-identifying information attached to the bag or the container may be read by the reader 11e also when the bag or the container is removed from the sterilizer, and the end time of the sterilization treatment should be recorded.

Thus, as illustrated by the table 440 shown in FIG. 5, information is input to columns 441 to 449, which store set ID, instrument ID, component ID, packaging material, sterilization method, sterilization condition, effective period, worker, and work date and time, respectively. The records of the work information to be recorded in the sterilization area A5 are not limited to the example of FIG. 5. The record of the work information to be recorded in the sterilization area A5 may include further detailed information. For example, the records of the work information obtained at the sterilization area A5 may include image data of the indicator after the sterilization treatment.

Here, a period of time for which the sterilization treatment remains effective is input into the column 447 that stores effective period.

The effective period of sterilization treatment may be determined from the sterilization method and the sterilization conditions. In this case, the relationship between sterilization method, sterilization conditions, and effective period may be recorded in advance in the master data of the system 10. It is also possible that the effective period of sterilization treatment may be determined from the sterilization method and the sterilization conditions. In that case, the relationship between packaging method, sterilization method, sterilization conditions, and effective period may be prerecorded in the master data of the system 10. For example, it is desirable that when the packaging method, the sterilization method, and the sterilization conditions are input at the sterilization area A5, the effective period should be accordingly input based on the master data.

The column 449 for work date and time may record, for example, the time at which the item-identifying information attached to the bag or the container has been read by the reader lie when the sterilization treatment was completed and the bag or the container was taken out of the sterilizer.

The processor 100 of the system 10 may be configured or programmed to collect the item-identifying information read by the readers 11a to 11f of the respective areas A1 to A6 and the work information associated with the item-identifying information. This enables the system 10 to collect the information of the instruments and the surgical instrument sets to be managed by the system 10 at the areas A1 to A6 and to collectively manage the information. In addition, it is possible to extract the work information indicating when, in which area, and what kind of work was performed for a certain instrument, in chronological order.

The third memory storage 103 of the system 10 stores a work history of an instrument identified by an instrument ID, in association with the instrument ID. The fourth memory storage 104 of the system 10 stores a work history of a surgical instrument set identified by a set ID in association with the set ID.

In this preferred embodiment, the processor 100 of the system 10 collects the work information that was recorded in the areas A1 to A6 from the respective control terminals 13a to 13f. This allows the processor 100 to store the work history of the instrument identified by the instrument ID in association with the instrument ID. This also allows the processor 100 to store the work history of the surgical instrument set identified by the set ID in association with the set ID.

Thus, the third memory storage 103 and the fourth memory storage 104 may be configured to collect the work information recorded at the areas A1 to A6 from the respective control terminals 13a to 13f. As a result, the information containing the work history of the instrument that is indicated by the instrument ID may be stored in association with the instrument ID. Likewise, the information containing the work history of the surgical instrument set that is identified by the set ID may be stored in association with the set ID. Thus, the work histories of the instruments that are to be stored in association with the instrument IDs do not need to be stored in a single database for each of the instrument IDs. Also, the work histories of the surgical instrument sets that are to be stored in association the set IDs do not need to be stored for each of the set IDs in a single database.

The system 10 extracts work information based on an instrument ID from the work information that is stored in the processor 100 and arranges the extracted work information in chronological order based on the work time, to obtain the work information of the instrument identified by the instrument ID appropriately. The system 10 also extracts work information based on a set ID from the work information that is stored in the processor 100 and arranges the extracted work information in chronological order based on the work time, to obtain the work information of the surgical instrument set identified by the set ID appropriately.

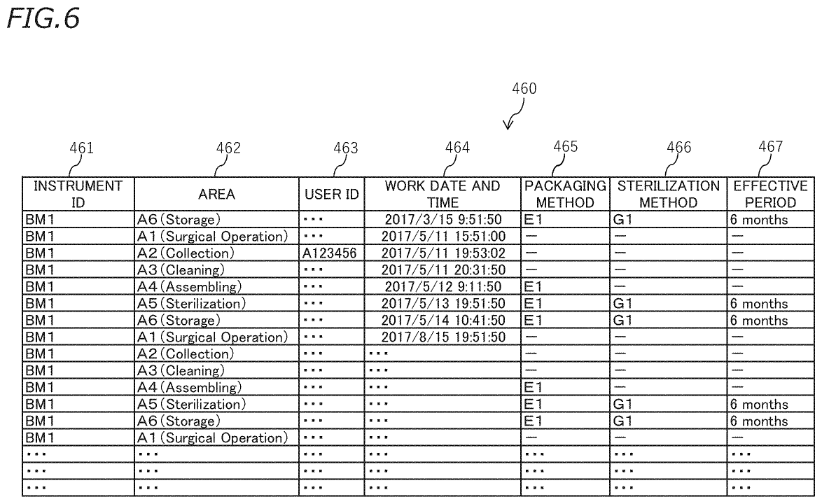

FIG. 6 illustrates an example of a table including data obtained by extracting the work history of a certain instrument. The table 460 shown in FIG. 6 contains items 461 to 467, which respectively indicate instrument ID, area (area ID), worker (user ID), work date and time, packaging method, sterilization method, and effective period, which are extracted consecutively to form a list. For a surgical instrument set as well, the work history can be extracted in a similar manner to that shown in FIG. 6. The items that are to be extracted for the work history are not limited to the example shown in FIG. 6. The work history may also contain all of the work times recorded at the areas A1 to A6. It is also possible that the work history may not contain all of the work times recorded at the areas A1 to A6. For example, it is possible to extract a predetermined portion of the work information from the work information recorded at the areas A1 to A6.

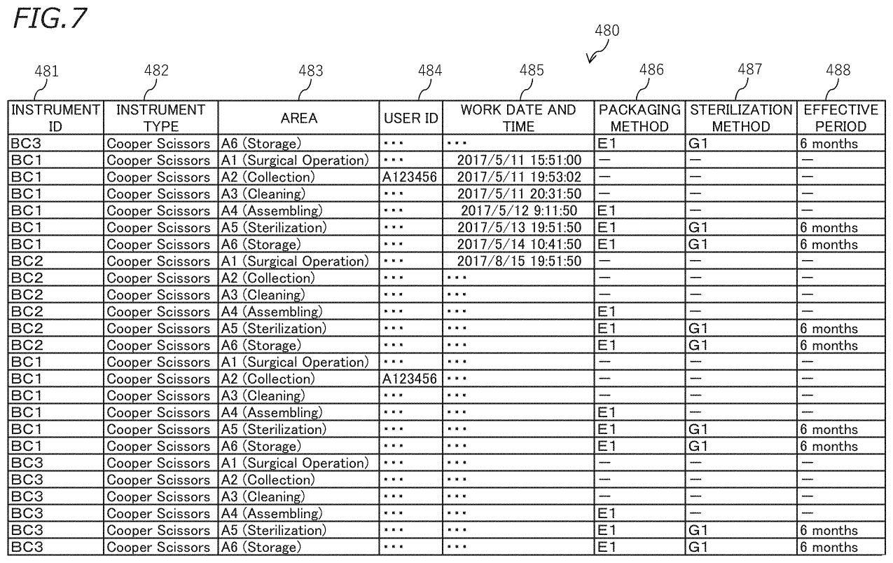

When a plurality of instruments of the same type are managed by the system 10, it is possible to extract the work histories of a plurality of instruments of the same type based on the type of instrument. FIG. 7 illustrates an example of a table of data in which the work histories of a plurality of instruments of the same type are extracted based on the type of instrument. The table 480 shown in FIG. 7 illustrates the results of extraction in which the work histories of a plurality of Cooper scissors have been extracted when, for example, the system 10 manages a plurality of Cooper scissors, which are individual surgical instruments not included in a surgical instrument set. Herein, three Cooper scissors (instrument IDs: BC1, BC2, BC3) are managed by the system 10. In FIG. 7, the work histories of the three Cooper scissors (instrument IDs: BC1, BC2, BC3) are extracted. The table 480 shown in FIG. 7 contains items 481 to 488, which respectively indicate instrument ID, instrument type, area (area ID), worker (user ID), work date and time, packaging method, sterilization method, and effective period, which are extracted consecutively to form a list.

In this preferred embodiment, the first processor 201 of the system 10 is configured or programmed to obtain the utilization rate of an instrument based on the work history of the instrument and a predetermined first calculation formula. The second processor 202 is configured or programmed to obtain the utilization rate of a surgical instrument set based on the work history of the surgical instrument set and a predetermined second calculation formula.

Herein, the utilization rate of an instrument is defined, for example, by the number of times the instrument has been used within a certain period of time. In this preferred embodiment, the utilization rate of an instrument may be defined by how many times the instrument underwent the cycle specified by the following procedure, surgical operation→collection (sorting)→cleaning→assembling (sealing)→sterilization→storage→surgical operation, within a certain period of time. Herein, how many times the instrument underwent the cycle may be obtained by counting the number of times the instrument was used in the surgical operation area A1, or by counting the number of times the instrument was cleaned in the cleaning area A3. Because the utilization rate of the instrument and the utilization rate of the surgical instrument set are obtained in this way, it is possible to judge how effectively the surgical instrument and the surgical instrument set are utilized based on the numerical values.

Herein, the first processor 201 may be configured or programmed to perform a process SA1 to identify an instrument A for which the utilization rate is to be obtained, and a process SA2 to set a period Z1 for which the utilization rate of the instrument A is calculated. The first calculation formula may be configured to obtain a count M1 of the number of times the instrument A has been used for the period Z1.

Here, the process SA1 identifies the instrument A. Here, the instrument A may be a specific instrument associated with an instrument ID. In this case, the computation of the utilization rate of the instrument A may obtain the number of times the specific instrument has been used for the period Z1. When the type of instrument is recorded as the information of the instrument, the first processor 201 may extract, for the instrument A, the instruments of the same type that are identified by the type of instrument. In this case, the computation of the utilization rate of the instrument A may obtain, for example, how many times the same type of instrument has been used on average for the period Z1. In other words, it is desirable to divide the number of times the same type of instrument was used, which was counted for the period Z1, by the number of the instruments of the same type, to obtain the arithmetic mean. The instrument A that is identified by the process SA1 is not limited to a specific instrument identified by an instrument ID, but may be any desired instrument.

The process SA2 may select a predetermined period for the period Z1, such as the last 6 months or the last 12 months, for the period Z1. It is also possible to input any desired period, such as from “a certain time” to “a certain time”. The first calculation formula may also be set as desired. It should be noted that the utilization rate may be changed as appropriate by depending on the input for the period Z1. It is desirable that the utilization rate can be changed appropriately by changing the period Z1.

The second processor 202 may be configured or programmed to perform a process SB1 to identify a surgical instrument set B for which the utilization rate is to be obtained, and a process SB2 to set a period Z2 for which the utilization rate of the surgical instrument set B is calculated. Here, it is desirable that the second calculation formula be configured to obtain a count M2 of the number of times the surgical instrument set B has been used for the period Z2. Here, the surgical instrument set B may be defined as a specific surgical instrument set associated with a set ID. In this case, the computation of the utilization rate of the surgical instrument set B may obtain, for example, the count of the number of times the specific surgical instrument set has been used for the period Z2. When the type of instrument is recorded as the information of the instrument, the second processor 202 may extract, for the surgical instrument set B, the surgical instrument sets of the same type that are identified by the type of surgical instrument set. In this case, the computation of the utilization rate of the surgical instrument set B may obtain, for example, how many times the same type of surgical instrument sets has been used on average for the period Z2. In other words, it is desirable to obtain the arithmetic mean by dividing the number of times the same type of surgical instrument set was used that was counted for the period Z2 by the number of the surgical instrument sets of the same type. Thus, the surgical instrument set B that is identified by the process SB1 is not limited to a specific instrument identified by a set ID, but may be any desired surgical instrument set. The period Z2 can be determined by following the above-described procedure of determining the period Z1 for the first processor 201, so the details will not be further described herein.

It should be noted that the process SB1 to identify the surgical instrument set B and the process SB1 to identify the surgical instrument set B are not limited to the examples described above. For example, for the plurality of instruments included in the surgical instrument sets, the first memory storage 101 may additionally store set IDs of the surgical instrument sets in association with the instrument IDs (see, for example, FIG. 3). In this case, the first processor 201 may be configured or programmed to extract an instrument ID associated with a set ID from among the instruments IDs assigned to the instruments to obtain the utilization rate of an instrument included in a surgical instrument set. This makes it possible to obtain the utilization rate of an instrument included in a surgical instrument set. In this case, the utilization rate of the instrument may be the average utilization rate of the instruments included in the surgical instrument sets, which is obtained by dividing the number of times the instruments included in the surgical instrument sets have been used by the number of the instruments included in the surgical instrument sets. It is also possible to extract an instrument that is not included in the surgical instrument sets. Consequently, it is also possible to obtain the utilization rate of an instrument not included in the surgical instrument sets.

For example, the first memory storage 101 may store information related to owners in association with the instrument IDs. The second memory storage 102 may store information related to owners in association with the set IDs. In this case, the system 10 may further include the third processor 203 configured or programmed to extract an instrument ID of an instrument or a set ID of a surgical instrument set, for each type of instrument, for each type of surgical instrument set, or for each owner. The third processor 203 is able to extract the instrument ID or the set ID based on the information related to owners that is associated with the instrument IDs or the set IDs. Herein, the owner information may mean information indicating, for example, a hospital or a medical department. For each of the instruments and the surgical instrument sets, it is possible to provide a plurality of items indicating the owner (see FIG. 3). In this case, the utilization rates of the instruments and the surgical instrument sets may be totaled for each owner. In other words, it is possible to obtain the utilization rates of the instruments for each hospital or for each medical department. Because the utilization rates of the instruments can be obtained for each hospital or for each medical department, it is possible to change the owner of the instrument from a hospital or a medical department with a low utilization rate to a hospital or a medical department with a high utilization rate. Also in that case, the system 10 may be configured or programmed to calculate the possession rate of instruments and surgical instrument sets for each owner. This allows the user to know how many instruments or surgical instrument sets the hospital or the medical department owns. This makes it easy to carry out management of the instruments and the surgical instrument sets that takes total optimization into consideration so that the instruments and the surgical instrument sets can be used efficiently.

The fourth processor 204 is configured or programmed to extract instrument IDs of an instrument based on the utilization rate of the instrument. In this case, the fourth processor 204 may be further configured or programmed to cause the display 12 to display the instrument IDs of the instrument which have been extracted based on the utilization rate.

The fifth processor 205 is configured or programmed to extract set IDs of a surgical instrument set based on the utilization rate of the surgical instrument set. In this case, the fifth processor 205 may be further configured or programmed to cause the display 12 to display the set IDs of the surgical instrument set which have been extracted based on the utilization rate of the surgical instrument set.

For example, the instruments and the surgical instrument sets that have a low utilization rate should be used preferentially so that variations in the frequency of use is able to be reduced among the instruments and the surgical instrument sets of the same type. That is, when an instrument or a surgical instrument set to be used in the surgical operation area A1 needs to be picked out at the storage area A6, an instrument or a surgical instrument set that has a low utilization rate may be extracted and picked out preferentially among the instruments or the surgical instrument sets of the same type.

In this case, the system 10 is also applicable to the case where the storage area A6 is what is called an automated warehouse, which includes a picking mechanism that sorts the instruments and the surgical instrument sets based on the instrument IDs and the set IDs to perform picking and receiving of goods. In this case, when picking out an instrument, one that has a low utilization rate may be picked out among the stored instruments, based on the utilization rate of the instrument. Furthermore, because the system 10 obtains the utilization rates of the instruments and the utilization rates of the surgical instrument sets, it is easy to recognize the presence of a surgical instrument set or a surgical instrument that has not been used at all for a certain period. In other words, because the system 10 is able to extract an instrument or a surgical instrument set that shows a utilization rate of zero, it is easy to recognize the presence of a surgical instrument set or a surgical instrument that has not been used at all for a certain period.

In this case, the system 10 may be configured or programmed to display the numbers of instruments and surgical instrument sets for each of utilization rates, non-use rate (the proportion of instruments or surgical instrument sets with a utilization rate of 0), and the like, in a bar graph or a pie chart, for example. The system 10 may also display the change of utilization rate of the instrument or the surgical instrument set in a line graph. This allows the user to recognize the utilization rate and the changes of utilization rate visually. As a result, the user is able to understand the utilization rate and the changes of utilization rate easily. Also in that case, the system 10 may be configured or programmed to create a summary or a graph for each owner, such as a hospital or a medical department, or for each type of instrument or surgical instrument set, for example.

It is also possible that the work histories of the instruments stored in the third memory storage 103 may include sterilization methods and sterilization conditions for the instruments. It is also possible that the work histories of the surgical instrument sets stored in the fourth memory storage 104 may include sterilization methods and sterilization conditions for the surgical instrument sets. In this case, the fifth memory storage 105 may store sterilization effective periods in association with the sterilization methods and the sterilization conditions. The sixth processor 206 may be configured or programmed to obtain an effective period of sterilization performed for the instrument or the surgical instrument set, based on at least the sterilization methods and the sterilization conditions that are stored in the fifth memory storage 105.

Here, examples of the sterilization methods include steam pressure sterilization (what is called an autoclave), dry heat sterilization, flame sterilization, boiling sterilization, free-flowing steam sterilization, ethylene oxide gas sterilization (what is called EOG sterilization), carbon dioxide gas sterilization, radiation sterilization, gamma-ray sterilization, ultraviolet sterilization, and formaldehyde sterilization. The term “sterilization conditions” means conditions adopted in any of these sterilization methods.

It is also possible that the effective period of sterilization may vary depending on the packaging method. If that is the case, it is also possible to take the packaging method into consideration. Herein, the packaging method may be specified by, for example, packaging materials and sealing methods.

It is also possible that the work histories of the instruments stored in the third memory storage 103 may include packaging methods, sterilization methods, and sterilization conditions for the instruments. It is also possible that the work histories of the surgical instrument sets stored in the fourth memory storage 104 may include packaging methods, sterilization methods, and sterilization conditions for the surgical instrument sets. The fifth memory storage 105 may store sterilization effective periods in association with the packaging methods, the sterilization methods, the sterilization conditions. The sixth processor 206 may be configured or programmed to obtain an effective period of sterilization performed for an instrument or a surgical instrument set, based on the sterilization methods and the sterilization conditions that are stored in the fifth memory storage 105. In the case where there are other items that affects the sterilization effective period (such as a storage method) other than the packaging methods, the sterilization methods, and the sterilization conditions, it is possible that additional conditions may be added to the master data appropriately so that such other items are taken into account to obtain the effective period of the sterilization.

The seventh processor 207 is configured or programmed to calculate a remaining expiration period for the sterilization effective period of one of the instruments or one of the surgical instrument sets. For example, the seventh processor 207 may be configured or programmed to obtain the sterilization effective period with reference to the end date and time of the sterilization treatment, to calculate the remaining expiration period. It should be noted that the remaining expiration period for the effective period may be calculated by any suitable method. The seventh processor 207 may be further configured or programmed to display remaining expiration periods calculated for each type of instrument or for each type of surgical instrument set on the display 12. The seventh processor 207 may be further configured or programmed to extract an instrument or a surgical instrument set based on the remaining expiration period. When this is the case, the seventh processor 207 may be further configured to display the instrument ID of the instrument or the set ID of the surgical instrument set ID, which has been extracted based on the remaining expiration period.

In this case, the calculated remaining expiration period for the sterilization effective period enables the worker is to easily recognize the remaining expiration period for the sterilization effective period for the instrument or the surgical instrument set. Because the remaining expiration period calculated for each of the instruments or the surgical instrument sets is displayed on the display 12, the worker is able to check the remaining expiration period for the sterilization effective period of the instrument or the surgical instrument set on the display 12.

For example, the remaining expiration period for the instrument or the surgical instrument set may be displayed in the form of a list. It is also possible that the instruments and the surgical instrument sets stored in the storage area A6 may be, for example, categorized by remaining expiration period and displayed in a bar graph or a pie chart. Furthermore, the system 10 may be configured or programmed to display a list of the instruments and the surgical instrument sets that are categorized by their remaining expiration period. For example, it is possible to obtain a list of the instruments and the surgical instrument sets that have a remaining expiration period of zero, i.e., that have reached so-called expired sterility. In this case, it is desirable that the list display the dates on which the instruments and the surgical instrument sets have reached the expired sterility. This enables the worker to know the number of days that have elapsed since the expired sterility. It is also possible that the system may be configured or programmed to display a list of the instruments and the surgical instrument sets that have a remaining expiration period of less than 1 month. The instruments and the surgical instrument sets that have a remaining expiration period of less than 1 month are considered as having higher priority to be used. The instruments and the surgical instrument sets that have reached a remaining expiration period of zero, i.e., that have reached the expired sterility, may be subjected to a sterilization treatment again. In this case, the system 10 may issue an alert warning the expired sterility, then total the counts of the number of times of re-sterilization for each of the re-sterilized instruments and materials, and display the counts of the number of times of re-sterilization on the displays 12a to 12g in a predetermined form (also referred to as a dashboard). Here, when re-sterilization is carried out a plurality of times, the process flow is as follows: surgical operation→collection→cleaning→assembling→sterilization→storage→re-sterilization→storage→re-sterilization. In this case, the sterilization that is performed at the first time after assembling is not considered as re-sterilization, so it is not included in “the count of the number of times of re-sterilization”.

For example, the system 10 may include an eleventh processor 211 configured or programmed to extract the instrument ID of an instrument that has been re-sterilized, based on the work histories of the instruments stored in the third memory storage 103. In this case, the eleventh processor 211 may be configured or programmed to obtain the count of the number of times the instrument with the extracted instrument ID has been re-sterilized.

The system 10 may further include a twelfth processor 212 configured or programmed to extract the set ID of a surgical instrument set that has been re-sterilized based on the work histories of the surgical instrument sets stored in the fourth memory storage 104. In this case, the twelfth processor 212 may be configured or programmed to obtain the count of the number of times the surgical instrument set with the extracted set ID has been re-sterilized.

Here, the process to extract the instrument ID of the re-sterilized instrument or the set ID of the re-sterilized surgical instrument set may be configured to extract the IDs of the instruments or the surgical instrument sets that have undergone a sterilization treatment a plurality of times at predetermined intervals without having been used at the surgical operation area A1.

Also, when the counts of the number of times the instruments or the surgical instrument sets with the extracted IDs have been re-sterilized were obtained, the system 10 may be configured or programmed to further extract an instrument or a surgical instrument set that has been re-sterilized a greater number of times than a predetermined number of times.

As a result, an instrument or a surgical instrument set is further extracted according to the count of the number of times of re-sterilization. Because the instruments or the surgical instrument sets that have been re-sterilized a greater number of times than a predetermined number of times, the trouble of looking for the instruments or the surgical instrument sets that have been re-sterilized a large number of times is reduced. In this case, the work information recorded at the collection area A6 may include information indicating the storage location of each of the instruments and the surgical instrument sets. This serves to further reduce the trouble of picking out the instruments or the surgical instrument sets that have been re-sterilized a large number of times. Furthermore, for the instruments or the surgical instrument sets that are re-sterilized without having been used, the number of the instruments or the surgical instrument sets may be reduced. For example, the system 10 may be configured or programmed to calculate, for each type of instrument or surgical instrument set, the proportion of the instruments and the surgical instrument sets that have been re-sterilized without having been used. It is also possible that the system 10 may be configured or programmed to calculate the optimum number of instruments or surgical instrument sets for each type of instrument or surgical instrument set, based on the proportion of the instruments or the surgical instrument sets that have been re-sterilized without having been used.

Herein, the proportion of the instruments or the surgical instrument sets that have been re-sterilized a greater number of times than a predetermined number is referred to as the “rate of re-sterilization”. The predetermined number may be preset in the system 10 or may be suitably set by the worker as desired. When the predetermined number is changed, the “rate of re-sterilization” changes accordingly.

The system 10 may also be configured or programmed to extract an instrument or a surgical instrument set that shows a high rate of re-sterilization, for example, for each type of instrument that is managed and for each type of surgical instrument set that is managed. In this case, the system 10 may extract instruments or surgical instrument sets in descending order of rate of re-sterilization. The system 10 may extract the instruments or surgical instrument sets that show a rate of re-sterilization higher than a predetermined rate.

When the numbers of the instruments and the surgical instrument sets are reduced according to the descending order of the number of times of re-sterilization, the rate of re-sterilization decreases. Moreover, the utilization rate of the instrument or the surgical instrument set improves, and the number of man-hours required for re-sterilization decreases.

For this reason, it is desirable that the system 10 be configured or programmed to calculate the rate of re-sterilization for each type of instrument or surgical instrument set when the number of the instruments or the surgical instrument sets is reduced according to the descending order of the number of times of re-sterilization. This enables the system 10 to provide simulated data showing how the utilization rate of an instrument or a surgical instrument set changes when how many instrument or surgical instrument set is reduced according to the descending order of the number of times of re-sterilization.

The system 10 may also be configured or programmed to calculate how many instruments or surgical instrument sets that show a large number of times of re-sterilization should be eliminated in order to keep the rate of re-sterilization to be a predetermined rate or higher. This enables the system 10 to provide the data that indicates how many instruments or surgical instrument sets should be reduced in order to keep the rate of re-sterilization higher than a predetermined rate.

Thus, the system 10 can obtain the optimum number of instruments or surgical instrument sets for each type of instrument or surgical instrument set. The system 10 may be configured or programmed to extract the instruments or the surgical instrument sets that have been re-sterilized a greater number of times than a predetermined number of times or to calculate the proportion of the instruments or the surgical instrument sets that have been re-sterilized a greater number of times than a predetermined number of times. Reducing the number of instruments or surgical instrument sets that shows a large number of times of sterilization actually serves to improve the utilization rate of the instrument or the surgical instrument set, to prevent the deterioration of the instrument or the surgical instrument set that has been re-sterilized without having been used, and to reduce the number of man-hours required for re-sterilization.

Furthermore, when the system 10 is configured or programmed to extract an instrument or a surgical instrument set based on the remaining expiration period, it is possible to extract, for example, the instruments or the surgical instrument sets that have reached a remaining expiration period of zero in the storage area A6. In addition, it is possible to pick out the instruments and the surgical instrument sets in an ascending order of remaining expiration period from among the instruments and the surgical instrument sets that have not yet reached a remaining expiration period of zero, when the instruments and the surgical instrument sets to be used in the surgical operation area A1 are selected in the storage area A6. When the instrument or the surgical instrument set that has been extracted based on the remaining expiration period is displayed on the display 12, the worker is allowed to pick out the instrument or the surgical instrument set while checking the screen of the display 12. This prevents the worker from picking out a wrong instrument or a wrong surgical instrument set. Moreover, the system 10 may also be configured or programmed to determine whether or not the instrument or the surgical instrument set that is picked out by the worker is correct, by reading the item-identifying information on the bag or the container enclosing the instrument or the surgical instrument set with the reader 11f.

The first memory storage may be configured to further store first classification information to classify the instruments by type in association with the instrument IDs. In this case, the system 10 may further include an eighth processor 208 that extracts one of the instrument IDs of the instruments based on the first classification information. The second memory storage 102 may further store second classification information to classify the surgical instrument sets by type in association with the instrument IDs. In this case, the system 10 may further include a ninth processor 209 that extracts one of the set IDs of the surgical instrument sets based on the second classification information.

Herein, the first classification information may be the type of instrument. The second classification information may be the type of surgical instrument set. It is possible to classify the instruments and the surgical instrument sets by type when there are a plurality of instruments of the same type or a plurality of surgical instrument sets of the same type. The process of extracting the instruments by the first classification information makes it possible to obtain, for example, the utilization rate of each type of instrument. Likewise, the process of extracting the surgical instrument sets by the second classification information makes it possible to obtain, for example, the utilization rate of each type of surgical instrument set.

As a result, it is possible to obtain the utilization rate of each type of instrument and the utilization rate of each type of surgical instrument set, for example. It is also possible to extract and classify the instruments and the surgical instrument sets for each of the areas A1 to A6, based on the most up-to-date work information of the work history. In this case, when the information is updated in real time, the status of the instrument or the surgical instrument set is obtained. For example, when an area is allocated for maintenance work such as repair work, the instrument or the surgical instrument set that belongs to the maintenance area may be extracted to extract the instrument or the surgical instrument set that is under maintenance. It is also possible to calculate the overall proportion of the instruments or the surgical instrument sets that are under maintenance. It is also possible that the instruments and the surgical instrument sets in the storage area A6 may be extracted to calculate the average storage dates of the instruments and the surgical instrument sets that are stored in the storage area A6. The average storage date may be calculated for each type of instrument or surgical instrument set. This offers an indicator that follows the utilization rate of instrument or surgical instrument set.

The system 10 herein causes the processor 100 to aggregate information to perform a predetermined process. Each of the control terminals 13a to 13f may be configured to acquire information from the processor 100 in a similar manner and to perform a similar process to that performed by the processor 100. In other words, each of the control terminals 13a to 13f may be configured to function as the system 10 on its own. The control terminals 13a to 13f may also be configured to bidirectionally communicate with the processor 100 to cooperate with the processor 100, and upon receiving the result computed by the processor 100, to display required information on the control terminals 13a to 13f. That is, it is also possible to construct a cloud-based system that uses the processor 100 as the host server. The cloud-based system makes it possible to obtain necessary information with client terminals that are connected, in a bidirectionally communicable manner, with the processor 100 serving as the host server. Each of the control terminals 13a to 13f, serving as a client terminal, may be configured to be a tablet terminal equipped with a camera. In this case, the camera fitted in the tablet terminal may be configured to function as a reading unit of each of the readers 11a to 11f. It is also possible that the screen of the tablet terminal may be configured to function as the displays 12a to 12g.

In the above-described preferred embodiments of the system 10, all the work histories from the surgical operation area A1 to the storage area A6 are aggregated in the processor 100. However, when calculating the utilization rate, or when calculating the remaining expiration period for the sterilization effective period, for example, all the work histories from the surgical operation area A1 to the storage area A6 are not always required, depending on the calculation formula and the calculation method. For example, when it is possible to calculate the utilization rate and the remaining expiration period for the sterilization effective period from the work information at the sterilization area A5 and the work information at the storage area A6, the system 10 does not necessarily need to manage the work information at the other areas. When that is the case, the system 10 may manage the work histories of the work information at the sterilization area A5 and the work information at the storage area A6 as the necessary information.

The system 10 may be constructed with only one computer, and it is possible to construct the system 10 in a stand-alone computer. For example, in a small medical facility such as a small-scale hospital, the areas from the surgical operation area A1 to the storage area A6 may be grouped into compact space. When this is the case, the system 10 may be constructed into a single computer.

In addition, the system 10 is able to collectively manage the information about the surgical instrument sets and the instruments that are used in a plurality of hospitals by, for example, gathering the information into the processor 100. In this case, the surgical operation area A1, the collection area A2, and the like are provided in each of the hospitals. In the system 10, it is desirable that the control terminals 13a to 13f in the respective areas A1 to A6 and the processor 100 be connected to each other so as to allow bidirectional communication with each other. It is also desirable that, by cloud computing technology, the work histories of the instruments and the surgical instrument sets as well as the utilization rates of the instruments and the surgical instrument sets, for example, be made available to an external terminal 21 connected to the system 10.

For example, as illustrated in FIGS. 1 and 2, the external terminal 21 may be connected to the system 10 through the communication network 14. The work histories of the instruments and the surgical instrument sets, the utilization rates of the instruments and the surgical instrument sets, and the like may be distributed from the system 10 to a predetermined terminal through the communication network 14 at predetermined timing. It is also desirable that data indicating the work histories of the instruments and the surgical instrument sets, the utilization rates of the instruments and the surgical instrument sets, and the like be displayed on the external terminal 21 in a predetermined form (also referred to as a dashboard).

Here, the external terminal 21 may be a terminal that is not installed in the areas A1 to A6. For example, the system 10 may be accessed from a terminal installed in, for example, a department of a hospital that takes charge of administrative work, accounting, inventory control, procurement of instruments and surgical instrument sets, and the like, which does not carry out such work as surgical operation and collection, to acquire the information of the instruments and the surgical instrument sets managed by the system 10. In addition, the external terminal 21 may be a portable terminal that is connectable with the communication network 14, such as a smartphone, a tablet computer, or a laptop computer. In this case, the system 10 can be accessed from any desired location to acquire the information of the instruments and the surgical instrument sets managed by the system 10.

The system 10 may incorporate a required authentication process and a required function restriction process to the external terminal 21 as needed. In that case, the system 10 may be configured or programmed to prepare a website that is browsable by a web browser installed in the external terminal 21. The system 10 may be configured or programmed to perform, through the prepared website, an authentication process for the external terminal 21 and provide a predetermined service in response to a request from the external terminal 21, for example.

For example, as illustrated in FIG. 1, the third processor 203 may be configured or programmed to cause, in response to a request from the external terminal 21 connected via the communication network 14, the external terminal 21 to display the work history of the instrument, the work history of the surgical instrument set, the utilization rate of the instrument, or the utilization rate of the surgical instrument set. When this is the case, it is desirable to cause the external terminal 21 to display the work history of the instrument, the work history of the surgical instrument set, the utilization rate of the instrument, or the utilization rate of the surgical instrument set for each of the instruments and the surgical instrument sets that have been extracted for each instrument, each surgical instrument set, or each owner.

In this case, the external terminal 21 may be configured to be able to specify an instrument, a surgical instrument set, or an owner, for example, through the web browser. In addition, the external terminal 21 may be configured to request the system 10 to enable the external terminal 21 to display the work history of the instrument, the work history of the surgical instrument set, the utilization rate of the instrument, or the utilization rate of the surgical instrument set, that has been extracted for the specified instrument, the specified surgical instrument set, or the specified owner.

The third processor 203 of the system 10 may be configured or programmed to cause, for example, in response to a request from the external terminal 21, the external terminal 21 to display, through the web browser, the external terminal to display the work history or the utilization rate of one of the instruments or the surgical instrument sets that is associated with the one of the instrument IDs or the set IDs having been extracted for each of the specified instruments, each of the specified surgical instrument sets, or each of the specified owners. Such a process can be embodied by applying cloud computing technology.

The fourth processor 204 of the system 10 may be configured or programmed to cause, for example, in response to a request from the external terminal 21 connected via the communication network 14, the external terminal 21 to display an instrument ID of the instrument that has been extracted based on the utilization rate of the instrument.

The fifth processor 205 of the system 10 may be configured or programmed to cause, for example, in response to a request from the external terminal 21 connected via the communication network 14, the external terminal 21 to display a set ID of the surgical instrument set that has been extracted based on the utilization rate of the surgical instrument set.

In this case, when a utilization rate to be extracted is input into the external terminal 21, for example, the system may extract instrument IDs of the instruments that have a utilization rate lower than the input utilization rate. The system 10 may also create a list containing the extracted instruments and the utilization rates of the instruments. Moreover, the list created by the system 10 may be displayed on the external terminal 21. The system 10 may also be configured or programmed to extract the instrument IDs of the instruments that have a utilization rate of zero (i.e., the instruments that have not been used for a certain period) and cause the external terminal 21 to display the list of the extracted instrument IDs. When this is the case, the utilization rate to be extracted and the period for which the utilization rate is calculated may be input into the external terminal 21. It is possible to configure the process of displaying the surgical instrument sets that have been extracted based on the utilization rate of the surgical instrument set in response to a request from the external terminal 21 in a similar manner.

In addition, the sixth processor 206 may be configured or programmed to cause, in response to a request from the external terminal 21 connected via the communication network 14, the external terminal 21 to display an effective period of sterilization performed for the instrument or the surgical instrument set. This allows the user to check the effective period of sterilization performed for the instrument or the surgical instrument set managed by the system 10 with the external terminal 21.

The seventh processor 207 may be configured or programmed to cause, in response to a request from the external terminal 21 connected via the communication network 14, the external terminal 21 to display a remaining expiration period for the effective period of sterilization performed for the instrument or the surgical instrument set. This allows the user to check the remaining expiration period for the effective period of sterilization performed for the instrument or the surgical instrument set managed by the system 10 with the external terminal 21.

The seventh processor 207 may be configured or programmed to cause, in response to a request from the external terminal 21 connected via the communication network 14, the external terminal 21 to display an instrument or a surgical instrument set that has been extracted based on the remaining expiration period. This allows the user to check the instrument or the surgical instrument set that has been extracted based on the remaining expiration period with the external terminal 21.

In this case, it is also desirable that the system 10 be configured or programmed to create a list containing the instruments and the surgical instrument sets as well as the remaining expiration period for the instruments and the surgical instruments, and cause the external terminal 21 to display the list. For example, the system 10 may create a list of the instruments and the surgical instrument sets that have a remaining expiration period of zero, a list of the instruments and the surgical instrument sets that have a remaining expiration period of equal to or less than 1 week, and a list of the instruments and the surgical instrument sets that have a remaining expiration period of equal to or less than 1 month. It is also possible that the system 10 may create a graph or the like that shows the number or proportion of the instruments and the surgical instrument sets that have a remaining expiration period of zero, the number or proportion of the instruments and the surgical instrument sets that have a remaining expiration period of equal to or less than 1 week, and the number or proportion of the instruments and the surgical instrument sets that have a remaining expiration period of equal to or less than 1 month, in a bar graph or a pie chart. Such a list and a graph may be created, for example, for each owner of the instruments or the surgical instrument sets, or for each type of instrument or surgical instrument set. It is also possible that the system 10 may be configured or programmed to create a graph that shows the change in the number or the proportion of the instruments and the surgical instrument sets that have a remaining expiration period of zero.

The eighth processor 208 may be configured or programmed to cause, in response to a request from the external terminal 21 connected via the communication network 14, the external terminal 21 to display an instrument that has been extracted based on the first classification information. This allows the user to check the instrument that has been extracted based on the first classification information from among the instruments managed by the system 10 with the external terminal 21.

In addition, the ninth processor 209 may be configured or programmed to cause, in response to a request from the external terminal 21 connected via the communication network 14, the external terminal 21 to display a surgical instrument set that has been extracted based on the second classification information. This allows the user to check the surgical instrument set that has been extracted based on the first classification information from among the instruments managed by the system 10 with the external terminal 21.

The system 10 may further include a tenth processor 210 configured or programmed to cause, in response to a request from the external terminal 21 connected via the communication network 14, the external terminal 21 to display the work histories of the instruments, the work histories of the surgical instrument sets, the utilization rates of the instruments, or the utilization rates of the surgical instrument sets. This allows the user to check the work histories and the utilization rates of the instruments and the surgical instrument sets that are managed by the system 10 with the external terminal 21.

The eleventh processor 211 may be configured or programmed to cause, in response to a request from the external terminal 21 connected via the communication network 14, the external terminal 21 to display the instrument ID of an instrument that has been re-sterilized. The twelfth processor 212 may be configured or programmed to cause, in response to a request from the external terminal 21 connected via the communication network 14, the external terminal 21 to display the set ID of a surgical instrument set that has been re-sterilized. This allows the user to check the instrument or the surgical instrument set that has been re-sterilized among the instruments and the surgical instrument sets managed by the system 10 with the external terminal 21. In this case, it is also desirable that the system 10 be configured or programmed to create a list containing the instruments and the surgical instrument sets that have been re-sterilized and the counts of number of times the instruments and the surgical instrument sets have been re-sterilized, and to cause the external terminal 21 to display the list. It is also possible that the proportion of the instruments and the surgical instrument sets that have been re-sterilized within a certain period may be displayed, for example, for each owner of the instruments and the surgical instrument sets, or for each type of instrument or surgical instrument set. The proportion of the surgical instrument sets and the surgical instrument sets that have been re-sterilized may be displayed in a graph, such as a bar graph or a pie chart. It is also possible that the system 10 may be configured or programmed to display the change in the proportion of the instruments or the surgical instrument sets that have been re-sterilized in a graph.

As described thus far, the system 10 may be configured or programmed to cause the external terminal 21 to display various types of data that can be provided by the system 10 in response to a request from the external terminal 21 connected via the communication network 14. Various types of data that can be provided by the system 10 are not limited to the examples described herein. Examples of the data that can be provided by the system 10 may include information stored in storage devices in the system 10, information computed or extracted through various kinds of processes performed by the system 10, and the graphs and lists created by the system 10.

Hereinabove, various preferred embodiments of the surgical instrument sets and instrument management systems proposed in this disclosure have been described, but it should be understood that the surgical instrument sets and instrument management systems proposed in this disclosure is not limited to the preferred embodiments described hereinabove. Various modifications and alterations may be made to the surgical instrument set and instrument management system disclosed herein. In addition, the features, structures, or steps described herein may be omitted where appropriate or may be combined in any suitable combinations, unless specifically stated otherwise.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A surgical instrument set and instrument management system comprising:
a first memory storage storing instrument IDs associated in a one-to-one correspondence with instruments;
a second memory storage storing set IDs associated in a one-to-one correspondence with surgical instrument sets, each of the surgical instrument sets including a plurality of instruments;
a third memory storage storing work histories of the instruments identified by the instrument IDs, the work histories being stored in association with the instrument IDs;
a fourth memory storage storing work histories of the surgical instrument sets identified by the set IDs, the work histories being stored in association with the set IDs;
a first processor configured or programmed to obtain a utilization rate of an instrument based on the work history of the instrument stored in the third memory storage and a predetermined first calculation formula; and
a second processor configured or programmed to obtain a utilization rate of a surgical instrument set based on the work history of the surgical instrument set stored in the fourth memory storage and a predetermined second calculation formula.

2. The surgical instrument set and instrument management system according to claim 1, further comprising:
a display; wherein
the first processor is further configured or programmed to cause the display to display the utilization rate of the instrument; and
the second processor is further configured or programmed to cause the display to display the utilization rate of the surgical instrument set.

3. The surgical instrument set and instrument management system according to claim 1, wherein the first processor is configured or programmed to implement:
a process (SA1) that identifies an instrument (A) for which the utilization rate is to be obtained; and
a process (SA2) that sets a period (Z1) for which the utilization rate of the instrument (A) is to be obtained; and
the first calculation formula is configured to obtain a count (M1) of a number of times the instrument (A) has been used for the period (Z1).

4. The surgical instrument set and instrument management system according to claim 1, wherein the second processor is configured or programmed to implement:
a process (SB1) that identifies a surgical instrument set (B) for which the utilization rate is to be obtained;
a process (SB2) that sets a period (Z2) for which the utilization rate of the surgical instrument set (B) is to be obtained; and
the second calculation formula is configured to obtain a count (M2) of a number of times the surgical instrument set (B) has been used for the period (Z2).

5. The surgical instrument set and instrument management system according to claim 1, wherein
for the plurality of instruments included in the surgical instrument sets, the first memory storage additionally stores set IDs of the surgical instrument sets in association with the instrument IDs; and
the first processor is configured or programmed to extract one of the instrument IDs associated with the set IDs from among the instruments IDs associated with the instruments, to obtain the utilization rate of one of the plurality of instruments included in one of the surgical instrument sets.

6. The surgical instrument set and instrument management system according to claim 5, wherein the first processor is further configured or programmed to extract one of the instrument IDs not associated with any one of the set IDs from among the instruments IDs associated with the instruments, to obtain the utilization rate of one of the instruments not included in any one of the surgical instrument sets.

7. The surgical instrument set and instrument management system according to claim 1, wherein
the first memory storage stores information related to owners in association with the instrument IDs; and
the second memory storage stores information related to owners in association with the set IDs;
the system further comprising:
a third processor configured or programmed to extract one of the instrument IDs or the set IDs for each type of the instruments, each type of the surgical instrument sets, or each of the owners.

8. The surgical instrument set and instrument management system according to claim 7, wherein the third processor is configured or programmed to cause, in response to a request from an external terminal connected via a communication network, the external terminal to display the work history or the utilization rate of one of the instruments or the surgical instrument sets that is associated with the one of the instrument IDs or the set IDs having been extracted for each type of the instruments, each type of the surgical instrument sets, or each of the owners.

9. The surgical instrument set and instrument management system according to claim 1, further comprising a fourth processor configured or programmed to extract one of the instrument IDs based on the utilization rate of the instrument.

10. The surgical instrument set and instrument management system according to claim 9, wherein the fourth processor is configured or programmed to cause, in response to a request from an external terminal connected via a communication network, the external terminal to display the one of the instrument IDs that has been extracted based on the utilization rate of the instrument.

11. The surgical instrument set and instrument management system according to claim 1, further comprising a fifth processor configured or programmed to extract one of the set IDs based on the utilization rate of the surgical instrument set.

12. The surgical instrument set and instrument management system according to claim 11, wherein the fifth processor is configured or programmed to cause, in response to a request from an external terminal connected via a communication network, to display the one of the set IDs that has been extracted based on the utilization rate of the surgical instrument set.

13. The surgical instrument set and instrument management system according to claim 1, wherein
the work histories of the instruments stored in the third memory storage include sterilization methods and sterilization conditions for the instruments; and
the work histories of the surgical instrument sets stored in the fourth memory storage include sterilization methods and sterilization conditions for the surgical instrument sets; and
the system further comprising:
a fifth memory storage storing sterilization effective periods in association with the sterilization methods and the sterilization conditions; and
a sixth processor configured or programmed to obtain an effective period of sterilization performed for one of the instruments or one of the surgical instrument sets, based on at least the sterilization methods and the sterilization conditions that are stored in the fifth memory storage.

14. The surgical instrument set and instrument management system according to claim 1, wherein
the work histories of the instruments stored in the third memory storage include packaging methods, sterilization methods, and sterilization conditions for the instruments; and
the work histories of the surgical instrument sets stored in the fourth memory storage include packaging methods, sterilization methods, and sterilization conditions for the surgical instrument sets;
the system further comprising:
a fifth memory storage storing sterilization effective periods in association with the packaging methods, the sterilization methods, and the sterilization conditions; and
a sixth processor configured or programmed to obtain an effective period of sterilization performed for one of the instruments or one of the surgical instrument sets, based on at least the packaging methods, the sterilization methods, and the sterilization conditions that are stored in the fifth memory storage.

15. The surgical instrument set and instrument management system according to claim 14, further comprising a seventh processor configured or programmed to obtain a remaining expiration period for the sterilization effective period of the one of the instruments or the one of the surgical instrument sets.

16. The surgical instrument set and instrument management system according to claim 1, wherein
the first memory storage further stores first classification information that classifies the instruments by type in association with the instrument IDs of the instruments;
the system further comprising:
an eighth processor configured or programmed to extract one of the instrument IDs of the instruments based on the first classification information.

17. The surgical instrument set and instrument management system according to claim 1, wherein
the second memory storage further stores second classification information that classifies the surgical instrument sets by type in association with the set IDs of the surgical instrument sets;
the system further comprising:
a ninth processor configured or programmed to extract one of the set IDs based on the second classification information.

18. The surgical instrument set and instrument management system according to claim 1, further comprising a tenth processor configured or programmed to cause, in response to a request from an external terminal connected via a communication network, the external terminal to display the work histories of the instruments, the work histories of the surgical instrument sets, the utilization rates of the instruments, or the utilization rates of the surgical instrument sets.

19. The surgical instrument set and instrument management system according to claim 1, further comprising an eleventh processor configured or programmed to extract one of the instrument IDs of an instrument that has been re-sterilized, based on the work histories of the instruments stored in the third memory storage.

20. The surgical instrument set and instrument management system according to claim 1, further comprising a twelfth processor configured or programmed to extract one of set IDs of a surgical instrument set that has been re-sterilized, based on the work histories of the surgical instrument sets stored in the fourth memory storage.
